(19) United States
(12) Patent Application Publication
(10) Pub. No.: US 2023/0349822 A1
(43) Pub. Date: Nov. 2, 2023

(54) GAS MEASURING DEVICE FOR DETERMINING THE CONCENTRATION OF AT LEAST ONE GAS COMPONENT IN A BREATHING GAS MIXTURE

(71) Applicant: Drägerwerk AG & Co. KGaA, Lübeck (DE)

(72) Inventors: Gerd PETER, Lübeck (DE); Bernd-Michael DICKS, Lübeck (DE); Robert JAHNS, Lübeck (DE); Martin KROH, Lübeck (DE); Lucas SCHNELLE, Lübeck (DE)

(21) Appl. No.: 18/303,795

(22) Filed: Apr. 20, 2023

(30) Foreign Application Priority Data
Apr. 28, 2022 (DE) 10 2022 110 302.1

(51) Int. Cl. G01N 33/497; G01N 21/03; G01N 21/31

(52) U.S. Cl. CPC G01N 21/31; G01N 21/0303; G01N 33/497; G01N 2201/0636; G01N 2201/08

(57) ABSTRACT

A gas measuring device (1000) for determining the concentration of a gas component in a breathing gas mixture includes a radiation source (1) with a illuminant (2) and a mirror arrangement (3) for emitting light radiation. A sample gas cuvette (5) is formed as a hollow body. A detector arrangement (15) with at least two bandpass filter elements (17, 18) and at least two detector elements (20, 21) receives the filtered light radiation. A control unit (42) is configured to detect signals from the detector elements (20, 21) and determine a concentration of a gas component in the breathing gas mixture. A light guide element (11) is provided in the form of a hollow body.

### CROSS REFERENCE TO RELATED APPLICATIONS

This application claims the benefit of priority under 35 U.S.C. §119 of German Application 10 2022 110 302.1, filed Apr. 28, 2022, the entire contents of which are incorporated herein by reference.

### TECHNICAL FIELD

The invention relates to a gas measuring device for determining the concentration of a gas component in a breathing gas mixture, for example an inhalation gas or an exhalation gas, of a living being.

### BACKGROUND

It is known to optically measure the composition of respiratory gases. The absorption of light in a certain wavelength range specific for the respective gas serves as a measure for the concentration of the respective gas. In this way, the concentrations of volatile anesthetic gases, carbon dioxide (CO₂) and nitrous oxide (N₂ O), among others, are measured in the breathing gas mixture of ventilated patients in the medical field. The breathing gas mixture may contain one or more so-called measured gases (measuring gases or sample gases), such as carbon dioxide, nitrous oxide or a variety of gaseous organic compounds such as methane, ethanol, acetone or volatile anesthetic gases, for example halothane, isoflurane, desflurane, enflurane, sevoflurane, i.e. those gas components whose concentration is to be determined by means of the gas measuring device. The anesthetic gases nitrous oxide, halothane, enflurane, isoflurane, methoxyflurane, sevoflurane, desflurane, xenon are used, for example, during surgical procedures to anesthetize patients. Acetone results as a possible metabolic product of patients, for example in the exhaled air of diabetics. Ethanol may be present in the exhaled air of intoxicated patients, for example.

In the long-term operation of these sensors, the problem arises again and again that errors occur in the displayed gas concentration due to changes in the emitted light power of the source or due to contamination in the light path. A typical solution is the use of thermal light sources (e.g. incandescent filament, filament, membrane emitters, LEDs), whose radiation has been optically suitably shaped and imaged onto a suitable detector by a sample gas cuvette (measured gas cuvette) filled with the gas mixture to be analyzed. Such a detector is able to detect light in different ranges of the infrared spectrum. Usually, this spectral separation is accompanied by a lateral (approximately perpendicular to the optical axis) spatial separation of the detector elements.

It is known to use parts of the detector to measure radiation in wavelength ranges where there is no absorption by the target gases. These measurements are usually used as a reference for assessing the total radiated light power or the attenuation due to contaminants in the beam path. However, a lateral spatial change in the light distribution can lead to errors in the gas measurement.

For this reason, additional means for lateral beam mixing are usually used in the beam path, but these reduce the light throughput, increase the overall size and/or cause additional costs. Solutions that can do without such means for beam mixing (for example Fabry-Perot detectors and filter wheel sensors), however, require the use of moving parts, which results in a sensitivity of the measurement to vibrations and shocks as well as mechanical wear. On the one hand, this is a considerable disadvantage for mobile use and, on the other hand, can lead to increased failure rates as well as a general limitation of the service life of the gas sensor technology.

Gas measuring devices are described, for example, in DE102010047159B4 and US2004238746A.

In US5739535B an infrared optical gas measuring device is described.

An infrared optical carbon dioxide sensor, a so-called IR carbon dioxide sensor, is known from US8399839B.

From US6571622B a combination sensor that is a combination of an infrared-optical carbon dioxide sensor with a flow sensor is known, which can be arranged in the main flow (main stream) in the respiratory gas path of a patient. From US2004238746A, and US2002036266A infrared optical carbon dioxide sensors are known, which can be arranged in the side stream in or at the respiratory gas path of a patient. US6954702B, US7606668B, US8080798B, US7501630B, US7684931B, US7432508B, and US7183552B disclose gas measurement systems for detecting gas concentrations in the side stream and main stream.

### SUMMARY

An object of the invention is therefore is to overcome the disadvantages of the prior art, in particular to provide a device for determining the concentration of a gas component in a breathing gas mixture, which enables good measurement quality at low manufacturing costs and with a small size.

This task is solved by a gas measuring device with features according to the invention. Advantageous further embodiments are indicated herein, some of which are explained in more detail with reference to the figures.

A gas measuring device for determining the concentration of a gas component in a breathing gas mixture, for example an inhalation gas or an exhalation gas, of a living being is presented.

A gas measuring device according to the invention comprises:
a source of radiation:
   a light source to emit light radiation in a wavelength range from 2.5 µm to 12.5 µm,
   a mirror arrangement
a sample gas cuvette (measured gas cuvette) in the form of a hollow body:
   an entry cuvette window,
   an exit cuvette window,
   a gas inlet,
   a gas outlet
a light guide element formed as a hollow body
a detector arrangement comprising:
   at least two bandpass filter elements,
   at least two detector elements, and
a control unit.

The radiation source is configured to emit light radiation in a wavelength range from 2.5 µm to 12.5 µm. The radiation source comprises a light source (comprising one or more illuminants) and a mirror arrangement. The wavelength range from 2.5 µm to 12.5 µm enables an infrared (IR) optical measurement of concentrations of nitrous oxide, carbon dioxide as well as different hydrocarbons such as volatile anesthetic gases or methane. For example, thermal light sources such as membrane emitters, incandescent filament or filament emitters or LEDs can be used as light sources in the radiation source.

The sample gas cuvette is configured as a hollow body. The sample gas cuvette is suitable and configured for guiding a light beam and for receiving a breathing gas mixture, gas or gas mixture.

The entrance cuvette window allows light radiation to enter the sample gas cuvette via an inlet opening of the sample gas cuvette.

The light radiation can exit the sample gas cuvette through an outlet opening of the sample gas cuvette and through the exit cuvette window.

The gas inlet is arranged on the sample gas cuvette and is provided for feeding the breathing gas mixture into the sample gas cuvette. The gas outlet is located on the sample gas cuvette and is provided for transporting the breathing gas mixture out of the sample gas cuvette.

The light guiding element is formed as a hollow body. The light guide element is configured to guide a light beam.

The light radiation can enter the light guide element through an inlet opening of the light guide element. The light radiation can exit the light guide element through an outlet opening of the light guide element.

The control unit is configured to detect signals from the at least two detector elements and to determine the concentration of the gas component in the breathing gas mixture from the signals from the at least two detector elements. In the control unit, measured values of the measuring channel and the reference channel are acquired and set in relation to each other. Usually, a quotient is formed from recorded measured values of the measuring channel to recorded measured values of the reference channel, which represents a measure of the concentration of the sample gas in the sample gas cuvette. The control unit may be connected to the radiation source, in particular the illuminant, via control lines. It may be suitable and configured to send control signals to the radiation source, in particular the illuminant.

The detector arrangement comprises at least two bandpass filter elements and at least two detector elements. The bandpass filter elements are suitable and configured for filtering the light radiation. The detector elements are configured for receiving and detecting the light radiation, in particular for detecting infrared radiation in infrared wavelength ranges in which absorption by gases, for example carbon dioxide, typically occurs.

The detector elements can be configured, for example, as semiconductor detectors, pyroelectric detectors (pyrodetectors), thermoelectric detectors (thermopiles, thermocouples), as thermal detectors (bolometers) or also as combinations thereof, for example as combinations of semiconductor detectors and thermal detectors.

The bandpass filter elements can, for example, be configured as optical interference filters in the form of interference layers on a substrate. These transmit light in a defined wavelength range. The bandpass filter elements are arranged and configured such that the light radiation emitted by the radiation source passes (directly or indirectly by means of a deflection) the bandpass filter elements before the detector elements. At least one of the at least two bandpass filter elements is configured to be optically transparent to a light radiation in a wavelength range which is absorbed by a measuring gas. The detector element arranged at this bandpass filter element constitutes the so-called measuring channel. At least one of the at least two bandpass filter elements is optically transmissive for a light radiation in a wavelength range which is not absorbed or only slightly absorbed by the measuring gas. The detector element arranged at this bandpass filter element represents the so-called reference channel. It may be provided that the detector arrangement comprises further bandpass filter-detector element combinations for determining the concentration of further measuring gases.

The detector arrangement may have an entrance window of the detector arrangement which protects the bandpass filter elements and detector elements from contamination. The entrance window of the detector arrangement may be infrared (IR) transparent. The entrance window may comprise a single crystal material. For example, the entrance window of the detector arrangement may be made of diamond, calcium fluoride, barium fluoride or potassium bromide. These have low refractive indices. Entrance windows made of silicon or germanium may have an anti-reflective coating.

In a preferred embodiment, the sample gas cuvette may have a reflective inner surface. The sample gas cuvette may be made of a reflective material, such as a metal having a polished inner surface. For example, the sample gas cuvette may be made of aluminum, the inner surface of which may have a polished finish. Alternatively, the inner surface of the sample gas cuvette may be coated with a reflective material.

In a preferred embodiment, the light guide element may have a reflective inner surface. The light guide element may be made of a reflective material, such as a metal or plastic having a polished inner surface. Alternatively, the inner surface of the light guide element may be coated with a reflective material. For example, the light guide element may be made of plastic and the inner surface may have a polished finish or a metal coating, such as gold or aluminum.

In a preferred embodiment, the sample gas cuvette extends in a tapered manner between the radiation source and the light guide element, and the light guide element extends in an expanded manner between the sample gas cuvette and the detector arrangement. The light radiation passes from the radiation source through the inlet cuvette window into the sample gas cuvette, then through the outlet cuvette window and finally through the light guide element to the detector arrangement.

In a preferred embodiment, the light guide element extends tapered between the radiation source and the sample gas cuvette, and the sample gas cuvette extends widened between the light guide element and the detector arrangement. The light radiation passes from the radiation source and through the light guide element via the inlet cuvette window into the sample gas cuvette, then through the outlet opening of the sample gas cuvette to the detector arrangement.

In preferred embodiments, a length LA of the light guide element may be a value in a range of 2.5 mm to 5.0 mm.

In a preferred embodiment, the sample gas cuvette extends as a hollow body in the form of a hollow truncated cone, that is, the sample gas cuvette is hollow truncated cone-shaped.

In the preferred embodiments of the sample gas cuvette with a typical embodiment as a hollow truncated cone, the light radiation enters the sample gas cuvette through the entry cuvette window and an inlet opening of the sample gas cuvette, which is usually substantially circular in shape.

In these typical embodiments, the light radiation exits the sample gas cuvette and an outlet opening of the sample gas cuvette, which is usually substantially circular in shape, and the outlet cuvette window.

In a preferred embodiment, the sample gas cuvette extends as a hollow body in the form of a truncated pyramid, i.e. the sample gas cuvette is hollow truncated pyramid shaped.

In a preferred embodiment, the light guide element extends as a hollow body in the form of a hollow truncated cone, that is, the light guide element is hollow truncated cone-shaped.

In a preferred embodiment, the light guide element extends as a hollow body in the form of a truncated pyramid, that is, the light guide element is in the shape of a hollow truncated pyramid.

In preferred embodiments, the sample gas cuvette and the light guide element are formed as hollow bodies in the shape of a truncated pyramid.

In preferred embodiments, the sample gas cuvette and/or the light guide element may be a hollow body in the form of a truncated cone or a truncated pyramid.

In preferred embodiments, the sample gas cuvette and the light guide element are formed as hollow bodies in the shape of a truncated cone.

In a preferred embodiment having a light guiding element formed as a truncated pyramid or a truncated cone, a pyramid angle or cone angle α of the light guiding element is a value in a range of 7.5° to 20.0°.

In a preferred embodiment, the sample gas cuvette extends in the form of a straight pyramid or a straight circular cone tapering between an inlet cuvette window and an outlet cuvette window, i.e. the cross-section of the sample gas cuvette is larger at the inlet cuvette window than at the outlet cuvette window. Preferably, the rotation axis of the sample gas cuvette is arranged on the optical axis. The cuvette windows can, for example each have a thickness of 0.5 mm to 2 mm.

In a preferred embodiment, the light guide element extends in the shape of a hollow truncated cone, in particular in the shape of a straight circular cone, expanding between the sample gas cuvette and the detector arrangement, i.e. the cross-section of the light guide element is smaller on the cuvette side than on the detector side or the inlet opening of the light guide element is smaller than the outlet opening of the light guide element. For example, the light guide element extends between the outlet cuvette window and the bandpass filter elements or the entrance window of the detector arrangement. For example, the light guide element extends between the radiation source and the inlet cuvette window.

The truncated pyramid or truncated cone configuration can compensate for a partial failure of the radiation source, since the truncated pyramid or truncated cone allows a uniform distribution of light of all relevant wavelength ranges on the measurement detector and the reference detector.

In preferred embodiments, dimensions and ratios between components may define the embodiments and design variations. The following embodiments show dimensions and dimensioning of the components of the gas measuring device as well as dimensional ratios of these components to each other.

In preferred embodiments, a length LA of the light guide element may be a value in a range of 2.5 mm to 5.0 mm.

In a preferred embodiment, a pyramid angle or a cone angle α of the light guiding element may be a value in a range 7.5° to 20.0°.

In preferred embodiments, an inlet opening DA2 of the light guide element may have a value in a range of 2.0 mm to 4.0 mm.

In a preferred embodiment, the pyramid angle or the cone angle α of the light guiding element may be smaller than or equal to the pyramid angle or cone angle β of the sample gas cuvette.

In a preferred embodiment, the pyramid angle or cone angle α may be defined by a ratio of the pyramid angle or cone angle α of the light guide element to a pyramid angle or cone angle β of the sample gas cuvette having a value in a range from 0.75 to 1.0.

In a preferred embodiment, the length of the light guide element LA may be formed to be shorter than or equal to a length of the sample gas cuvette LB.

In a preferred embodiment, the length of the light guide element LA may be defined by a ratio of the length of the light guide element LA to the length of the sample gas cuvette LB having a value in a range of 0.3 to 1.0.

In a preferred embodiment, the outlet opening DB2 of the sample gas cuvette may be larger than or equal to the inlet opening DA2 of the light guide element.

In a preferred embodiment, the outlet opening DB2 of the sample gas cuvette may be defined by a ratio of the outlet opening DB2 of the sample gas cuvette to the inlet opening DA2 of the light guide element having a value in a range from 1.0 to 2.0.

In a preferred embodiment, the inlet opening DA2 of the light guiding element may be formed smaller than the outlet opening DA1 of the light guiding element.

In a preferred embodiment, the inlet opening DA2 may be defined by a ratio of the inlet opening DA2 of the light guide element to the outlet opening DA1 of the light guide element having a value in a range of 0.3 to 0.9.

In a preferred embodiment, the inlet opening DB1 of the sample gas cuvette may be larger than the outlet opening DB2 of the sample gas cuvette.

In a preferred embodiment, the inlet opening DB1 of the sample gas cuvette may be defined by a ratio of the inlet opening DB1 of the sample gas cuvette to the outlet opening DB2 of the sample gas cuvette having a value in a range from 1.2 to 1.8.

In a preferred embodiment, the outlet opening DC of the mirror arrangement may be larger than or equal to the inlet opening DB1 of the sample gas cuvette.

In a preferred embodiment, the outlet opening DC of the mirror arrangement may be defined by a ratio of the outlet opening DC of the mirror arrangement to the inlet opening DB1 of the sample gas cuvette having a value in a range from 1.0 to 2.0.

In a preferred embodiment, a ratio of the outlet opening DC of the mirror arrangement to the length LC of the mirror arrangement may be a value in a range from 0.53 to 1.88.

In one or more embodiments, one or more silicon windows may be arranged on the light guide element in the beam path. For example, an inlet silicon window may be arranged on the light entrance side of the light guide element, i.e. on the sample gas cuvette side or on the radiation source side. Furthermore, an outlet silicon window may be arranged on the light exit side of the light guide element, i.e. on the detector side. In this case, 1 mm of (possibly cumulative) silicon thickness replaces about 3.4 mm of the length of the light guide element, so that the ratio of the length of the light guide element to the sample gas cuvette length is reduced accordingly. The use of one or more silicon windows allows a further reduction in the overall length of the gas sensing device. An inlet silicon window or an outlet silicon window or both an inlet silicon window and an outlet silicon window may be arranged on the light guiding element. The silicon windows may be configured as single crystal silicon wafers. It may be provided that a silicon window has a thickness of at least 0.5 mm. It may further be provided that the cumulative thickness of the silicon windows is at least 0.5 mm.

It may further be provided that the inlet silicon window forms or replaces the outlet cuvette window. It may then be provided that the inlet silicon window has a coating on the inside of the sample gas cuvette, in particular an anti-reflective coating, a biocompatible coating and/or an anesthetic gas-resistant coating. It may further be provided that the outlet silicon window forms or replaces the entrance window of the detector arrangement.

It may be provided that the entry silicon window and the outlet silicon window gas-tightly seal the light guide element, so that the penetration of parasitic CO₂ and contaminants is impeded or prevented. Suitable connections can be formed in particular bonding, potting, welding (in particular ultrasonic welding) connections. Sealing elements, in particular O-rings, may be provided.

In a preferred embodiment, an inlet silicon window may be disposed on the light entrance side of the light guide element.

In a preferred embodiment, an outlet silicon window may be disposed on the light exit side of the light guide element.

In a preferred embodiment, an optical path outside the sample gas cuvette may be reduced by a value in a range of 2.0 to 4.0 times the cumulative thicknesses of the silicon windows.

In a preferred embodiment, the length of the light guide element LA may be reduced by a value in a range of 2.0 to 4.0 times the cumulative thicknesses of the silicon windows.

In a preferred embodiment, the ratio of the length of the light guide element LA to the length LB of the sample gas cuvette may be reduced according to the cumulative thicknesses of the silicon windows.

Further embodiments indicate practicable design variants of the gas measuring device - in some cases with associated dimensioning of the individual components.

It may be provided that the axis of rotation of the light guiding element is arranged on the optical axis.

In one or more embodiments, a membrane emitter (membrane radiator) may be provided. In one or more embodiments, an LED may be provided. Preferably, the illuminant and the mirror arrangement are arranged and configured relative to each other such that the light emission from the radiation source, in particular along the optical axis, is in the direction of the detector arrangement. The mirror arrangement, often referred to as a reflector, may be arranged and configured such that the light radiation is directed towards the detector elements as efficiently as possible. The mirror arrangement may have a reflective inner surface. The mirror arrangement may be configured as a paraboloid or a paraboloid frustum, in particular with a circular cross-section. The mirror arrangement can also be configured as a so-called Winston-Cone (compound parabolic concentrator). Cone sections, ellipsoids, hyperboloids and mixed forms are also possible. It may be provided that the axis of rotation of the mirror arrangement is arranged on the optical axis. However, it may also be provided that the axis of rotation of the mirror arrangement is arranged off-axis (i.e. not on the optical axis). It may be provided that the outlet opening of the mirror arrangement is 0.53 times to 1.88 times the length of the mirror arrangement. In particular, it may be provided that the outlet opening and the length of the mirror arrangement are the same. For example, the mirror arrangement may have an exit radius of 1.2 mm to 1.6 mm, in particular 1.4 mm. The length of the mirror arrangement may be, for example, 1.7 mm to 4.5 mm, in particular 2.8 mm. The mirror arrangement can be made, for example, of metal with a well-reflecting, for example polished, surface, for example polished aluminum, brass or steel, as a turned part of steel, in particular coated with gold, or of coated plastic, for example coated with aluminum or gold.

The radiation source may comprise an exit window on the light exit side, which protects the radiation source against contamination. The exit window of the radiation source may be, for example, 0.5 mm to 1 mm thick. The exit window of the radiation source may be infrared (IR) transparent. It may be made of a monocrystalline material. For example, the radiation source exit window may be made of diamond, calcium fluoride, barium fluoride or potassium bromide. These have low refractive indices. Exit windows made of silicon or germanium may have an anti-reflective coating. It may be provided that the exit angle from the reflector is not more than 28°, in particular 26° to 27.7°, in particular 26.5°.

It may be provided that the inlet cuvette window and the outlet cuvette window seal the sample gas cuvette in a gas-tight manner so that the breathing gas in the sample gas cuvette cannot be contaminated by external influences (for example, incoming ambient gas). Suitable connections may be, for example, bonding, potting, welding (in particular ultrasonic welding) connections. Sealing elements, in particular O-rings, may be provided. It may be provided that the inlet cuvette window replaces the outlet window of the radiation source. The cuvette windows may be transparent to infrared radiation. They may, for example, be made of a monocrystalline material, for example barium fluoride or calcium fluoride.

Due to the contact with respiratory gases, the sample gas cuvette, the cuvette windows and the connections are preferably manufactured in such a way that the surfaces are biocompatible, in particular moisture-resistant and non-toxic.

It may further be provided, in particular if the breathing gas mixture contains anesthetic gases, that the sample gas cuvette, the cuvette windows as well as their connection are configured to be anesthetic gas resistant, i.e. the materials coming into contact with the anesthetic gas do not react with the anesthetic gas and are not changed or attacked by the contact. Anesthetic gas-resistant materials are, for example, calcium fluoride or barium fluoride.

The sample gas cuvette can, for example, be made of metal, e.g. aluminum, steel, brass, and in particular have a polished inner surface. Alternatively, the sample gas cuvette can also be made of coated, in particular metal-coated, plastic.

Furthermore, it may be provided that the sample gas cuvette has a measuring volume that is as small as possible so that a breath-resolved measurement is possible. For example, the measurement volume may be 127 mm³ to 142 mm³, in particular 113 mm³. The sample gas cuvette may have a length of 5.8 mm, for example. The entry radius, i.e. the light entry side, radiation source side radius of the sample gas cuvette may be, for example, 2.95 mm to 3.25 mm, in particular 3.25 mm. The exit radius, i.e. the light exit side, detector side radius of the sample gas cuvette may be, for example, 2 mm to 2.3 mm, in particular 2 mm. It may be provided that the inlet opening of the sample gas cuvette is about 1.28 times to 1.63 times, in particular 1.62 times, the outlet opening of the sample gas cuvette. The opening angle or the cone angle of the truncated cone of the sample gas cuvette may be, for example, 12.2° to 16.4°, in particular 12.2°. It may be provided that the outlet opening of the mirror arrangement (reflector) is at most twice as large as the inlet opening of the sample gas cuvette. In particular, it may be provided that the outlet opening of the mirror arrangement is 0.37 times to 0.54 times the inlet opening of the sample gas cuvette, in particular 0.43 times. It may be provided that the length of the light guide element is shorter than the sample gas cuvette length. In particular, it may be provided that the length of the light guide element is 0.48 times to 0.83 times, in particular 0.65 times, the measurement gas cuvette length. It may further be provided that the light guide element is 2.8 mm to 4.8 mm long, in particular 3.8 mm long. It may be further provided that the taper angle of the light guiding element is less than/equal to the taper angle of the sample gas cuvette. It may further be provided that the cone angle of the light guiding element is 0.57 times to 0.98 times, in particular 0.97 times, the cone angle of the sample gas cuvette. It may further be provided that the cone angle of the light guiding element is 9.5° to 16°, in particular 11.9°. The advantageous configuration of the cone angle of the light guiding element may contribute to reducing divergence in the beam path. It may further be provided that the inlet opening of the light guiding element is 0.5 times to 0.77 times, in particular 0.65 times, the outlet opening of the light guiding element. Furthermore, it may be provided that the outlet opening of the sample gas cuvette is larger than the inlet opening of the light guide element. In particular, it may be provided that the outlet opening of the sample gas cuvette is at most twice the inlet opening of the light guiding element. In particular, the outlet opening of the sample gas cuvette may be 1.18 times to 1.77 times, in particular 1.33 times, the inlet opening of the light guide element. It may be provided that the inlet opening of the light guide element is 2.6 mm to 3.4 mm, in particular 3 mm. It may further be provided that the outlet opening of the light guiding element is 4.4 mm to 5.2 mm, in particular 4.6 mm. In one or more embodiments, an aperture may be provided along the optical axis between the sample gas cuvette and the light guide element. In this regard, it may be provided that the aperture has an aperture diameter (area size) that is smaller than the inlet opening of the light guiding element. In particular, the aperture may have an aperture diameter of 2.5 to 3.3 mm, in particular 2.8 mm.

The control unit may be connected to the detector elements via measurement signal lines. It may be suitable and configured to receive measurement signals from the detector elements. It may further be configured to process the measurement signals from the detector elements. The control unit may comprise signal processing components such as microcontrollers, amplifiers, analog-to-digital converters. The control unit may be suitable and configured for activation, in particular for controlling the operation of the radiation source. The control unit may also comprise a closed-loop control. The control unit may comprise any type of influencing (controlling) configuration. The control unit may further comprise an interface to a display unit. The control unit may be suitable and configured to send an output signal to the display unit.

The invention is based on the concept of inserting a suitable light path between the sample gas cuvette and the detector. In the solution presented within the scope of the present invention, the beam path with beam shaping is extended outside the sample gas cuvette by means of a light guide element. In this way, effective beam mixing, high light throughput, good symmetry of the light distribution with almost constant intensity and angle distribution, and lower sensitivity to contamination can be achieved, while at the same time keeping manufacturing costs low and the overall length short. The use of costly additional optical elements can be dispensed with. Thus, the present gas measuring device is compact, light beam efficient, cost effective and provides high measurement quality.

In the following, the invention will be explained in more detail with reference to the embodiments. However, the invention is not limited to the embodiments presented herein; rather, combinations of the embodiments shown — even if not explicitly shown in detail — are also encompassed by the concepts of the invention. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a gas measuring device according to a first embodiment;

FIG. 2 is a schematic view showing a second embodiment;

FIG. 3 is a schematic view showing a third embodiment;

FIG. 4 is a schematic view showing a fourth embodiment;

FIG. 5 is a schematic view showing a fifth embodiment;

FIG. 6 is a schematic view showing a sixth embodiment with an alternatively configured gas measuring device; and

FIG. 7 is a schematic view showing aspects of FIGS. 1-6 with dimension designations.

### DESCRIPTION OF PREFERRED EMBODIMENTS

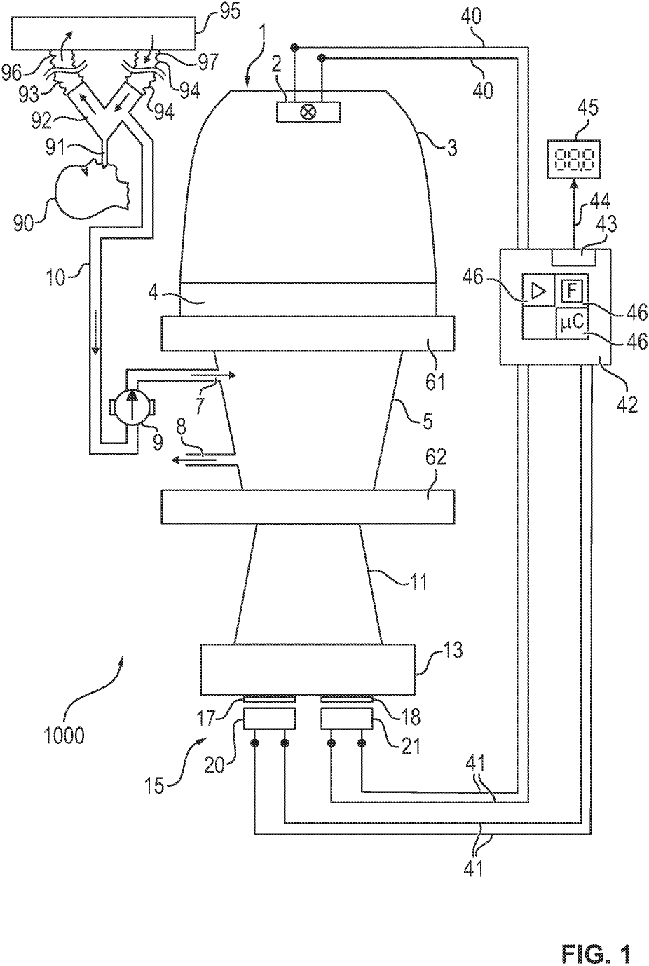

FIG. 1 shows a schematic representation of a gas measuring device 1000 according to a first embodiment and a patient 90 connected to the gas measuring device 1000 via a ventilator 95. The gas measuring device 1000 comprises a radiation source 1, a sample gas cuvette (sample gas cuvette) 5, a light guiding element 11, a detector arrangement 15 and a control unit 42.

A light radiation or light propagation passes, starting from the radiation source 1, through the entrance cuvette window 61, the cuvette 5, the outlet cuvette window 62, light guide element 11 and then reaches the detector arrangement 13. The radiation source 1 comprises a light source (illuminant) 2, a mirror arrangement 3 and an exit window 4 of the radiation source 1.

In alternative embodiments, the exit window may be omitted. The illuminant or light source 2 is exemplarily configured here as an LED (light emitting diode). However, in alternative embodiment examples, other light sources or radiation sources may be used. The illuminant emits light radiation in a wavelength range 2.5 µm to 12.5 µm. The mirror arrangement 3 is configured as a Winston-Cone (compound parabolic concentrator). However, in alternative embodiments, the mirror arrangement may have a different shape. The axis of rotation of the mirror arrangement 3 is arranged on the optical axis. The mirror arrangement has a length 31, LC and an outlet opening 35, DC. Adjacent to the outlet opening 35, DC of the mirror arrangement 3 is the exit window 4 of the mirror arrangement. The illuminant 2 and the mirror arrangement 3 are arranged relative to each other in such a way that the light radiation from the radiation source is emitted along an optical axis through the exit window 4 of the radiation source 1 in the direction of the detector arrangement 15.

In this FIG. 1, the sample gas cuvette 5 is hollow frustoconical in shape and has an inlet opening 52, DB1, an outlet opening 53, DB2, a measurement sample gas cuvette length 51, LB, and a cone angle 54, β.

The measurement sample gas cuvette 5 is bounded on the side of its inlet opening 52, DB1 by an inlet cuvette window 61 and is closed in a gas-tight manner. The light radiation can enter the sample gas cuvette through the inlet cuvette window 61. The sample gas cuvette 5 is bounded on the side of its outlet opening 53, DB2 by an outlet cuvette window 62 and closed in a gas-tight manner. The light radiation can exit the sample gas cuvette through the outlet cuvette window 62. The light inlet side, i.e. on the side of the radiation source 1, the inlet opening 52, DB1 of the sample gas cuvette 5 is larger than the light outlet side, i.e. on the side of the detector arrangement 15, the outlet opening 53, DB2 of the sample gas cuvette 5 – the sample gas cuvette thus has a hollow truncated cone shape tapering along the optical axis. The axis of rotation of the hollow frustoconical sample gas cuvette is arranged on the optical axis. The sample gas cuvette has a gas inlet 7 through which a breathing gas mixture can flow into the sample gas cuvette. The breathing gas mixture may comprise one or more sample gases, the concentration of which is to be determined. Furthermore, the sample gas cuvette comprises a gas outlet 8 through which gas present in the sample gas cuvette can flow out of the sample gas cuvette. For example, a patient 90 may be connected to a ventilator 95 at the gas inlet 7. FIG. 1 shows an endotracheal tube 91 attached to the patient 90, which is connected to the ventilator 95 via a connecting element (Y-piece) 92. Thereby, an expiratory ventilation tube 93 and an expiratory gas inlet 96 lead from the connecting element 92 into the ventilator 95, while an inspiratory gas outlet 97 and an inspiratory ventilation tube 94 lead out of the ventilator 95 back to the connecting piece 92. Connected to the inspiratory ventilation tube 94 is a sample gas line 10 — often referred to as a “sample line” — which leads to the gas inlet 7 of the sample gas cuvette 5 via a pump 9. Alternatively, the sample gas line 10 may also be connected elsewhere on the ventilation system, for example to the expiratory ventilation tube 93 or to both ventilation tubes 93, 94, depending on whether a gas concentration of an inspiratory gas and/or an expiratory gas is to be measured or whether comparative values between inspiratory and expiratory gas are also to be determined.

The detector arrangement 15 includes an entrance window 13, a first bandpass filter 17, a second bandpass filter 18, a measurement detector 20, and a reference detector 21. In alternative embodiments, the entrance window may be omitted.

The bandpass filter elements 17, 18 are each configured to pass only a particular wavelength range of light radiation emitted by the radiation source 1. The first bandpass filter 17 and the measurement detector 20 form a measurement channel. In this case, the first bandpass filter is configured to pass only the wavelength range of the light radiation that is absorbed by the measurement gas. The second bandpass filter 18 and the reference detector 21 form a reference channel. In this case, the second bandpass filter is configured to transmit only the wavelength range of light radiation that is not (or only slightly) absorbed by the sample gas. In alternative embodiments, the detector arrangement may comprise further bandpass filter-detector element combinations for determining the concentration of further sample gases.

The control unit 42 comprises components for signal processing 46, for example microcontrollers, amplifiers, analog-to-digital converters. The control unit 42 is connected to the detector elements 20, 21 via measurement signal lines 41 and to the radiation source 1 by means of control lines 40. The control unit receives measurement signals from the detector elements 20, 21 and uses them to calculate the concentration of the sample gas or gases. The control unit 42 further comprises an interface 43 via which the control unit 42 can send an output signal 44 to a display unit 45.

The light guide element 11 is disposed between the outlet cuvette window 62 and the entrance window 13 of the detector arrangement 15. In this FIG. 1, the light guide element 11 is hollow frustoconical in shape and has an inlet opening 113, DA2, an outlet opening 112, DA1, a length 111, LA, and a cone angle 114, α. The inlet opening 113, DA2 of the light guiding element 11 is smaller than the outlet opening 112, DA1 of the light guiding element 11 – the light guiding element 11 thus has a hollow frustoconical shape widening in the direction of the detector arrangement.

In the embodiment example according to FIG. 1, the light radiation from the radiation source 1 passes successively through the exit window 4 of the radiation source 1, the inlet gas cuvette window 61, the sample gas cuvette 5, the outlet cuvette window 62, the light guide element 11, the inlet window 13 of the detector arrangement 15, the bandpass filters 17, 18 and strikes the detector elements 20, 21.

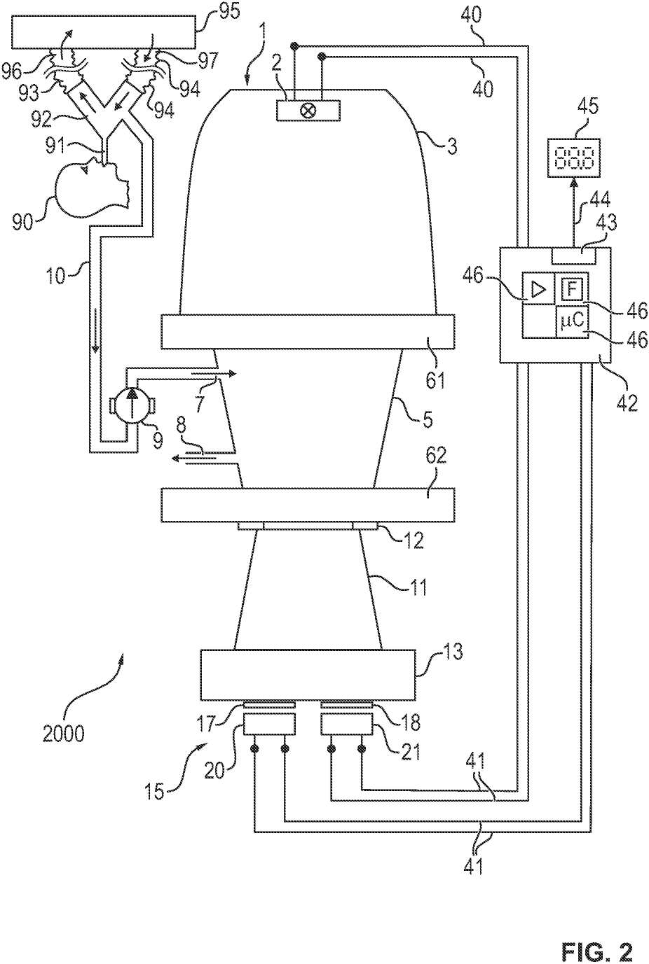

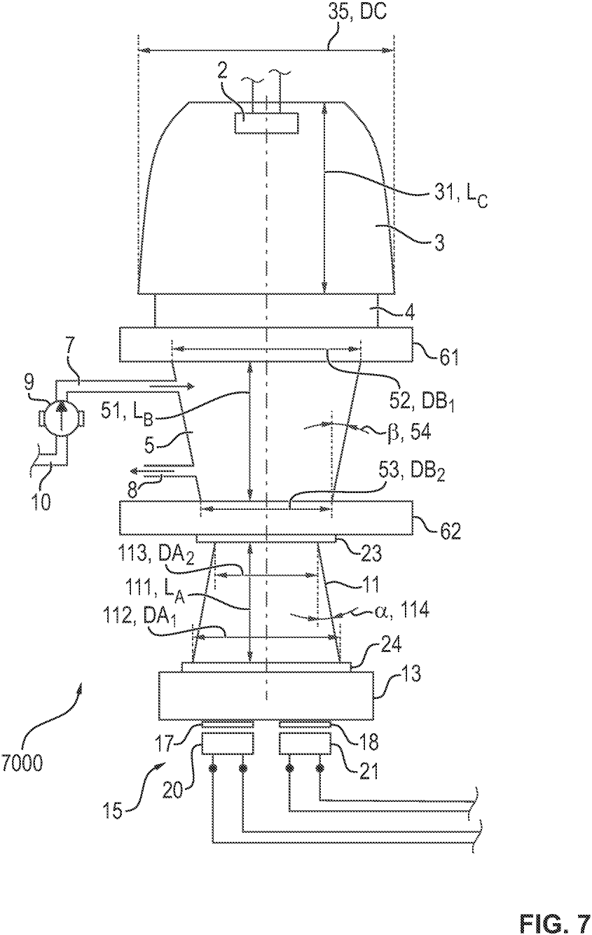

In alternative embodiments of the radiation source 1 with illuminant 2 and mirror arrangement 3 — as shown for example in FIG. 2 — the exit window 4 can be omitted. In alternative embodiments of the detector arrangement 15, the entrance window 13 may be omitted. In this embodiment example according to FIG. 1 and with reference to the dimension designations in FIG. 7, the following dimensions are provided: The mirror arrangement 3 has an outlet opening 35, DC of 2.4 mm to 3.2 mm, preferably 2.8 mm, and a length 31, LC of 1.7 mm to 4.5 mm, preferably 2.8 mm. The ratio of length 31, LC to outlet opening 35, DC is preferably 1:1. The sample gas cuvette 5 has an inlet opening 52, DB1 of 5.9 mm to 6.5 mm, preferably 6.5 mm, an outlet opening of 4 mm to 4.6 mm, preferably 4 mm, and a length of preferably 5.8 mm. The cone angle 54, β of the sample gas cuvette 5 is preferably 12.2° to 16.4°, preferably 12.2°. The ratio of the inlet opening 52, DB1 of the sample gas cuvette 5 to the outlet opening 53, DB2 of the sample gas cuvette 5 is preferably 1.625:1. The light guide element 11 has an inlet opening 113, DA2 of 2.6 mm to 3.4 mm, preferably 3 mm, an outlet opening 112, DA1 of 4.4 mm to 5.2 mm, preferably 4.6 mm, and a length 111, LA of 2.8 mm to 4.8 mm, preferably 3.8 mm. The taper angle 114, α of the light guide element 11 is preferably from 9.5° to 16°, preferably 11.9°. The ratio of the inlet opening 113, DA2 of the light guiding element 11 to the outlet opening 112, DA1 of the light guiding element 11 is preferably 0.65:1. In alternative embodiments, other dimensions may of course be provided.

FIG. 2 shows a schematic representation of a gas measuring device 2000 according to a second embodiment. Therein, the same elements are designated with the same reference numerals as in FIG. 1. For illustration purposes, in this embodiment the illuminant 2 is configured as a membrane emitter, and the exit window 4 (FIG. 1) of the radiation source 1 has been omitted. Another illuminant, for example LED, can also be used. FIG. 2 also shows an aperture 12 which is arranged in front of the light guide element 11. The aperture 12 has an opening dimension (diameter) which is smaller than the inlet opening 113, DA2 of the light guiding element.

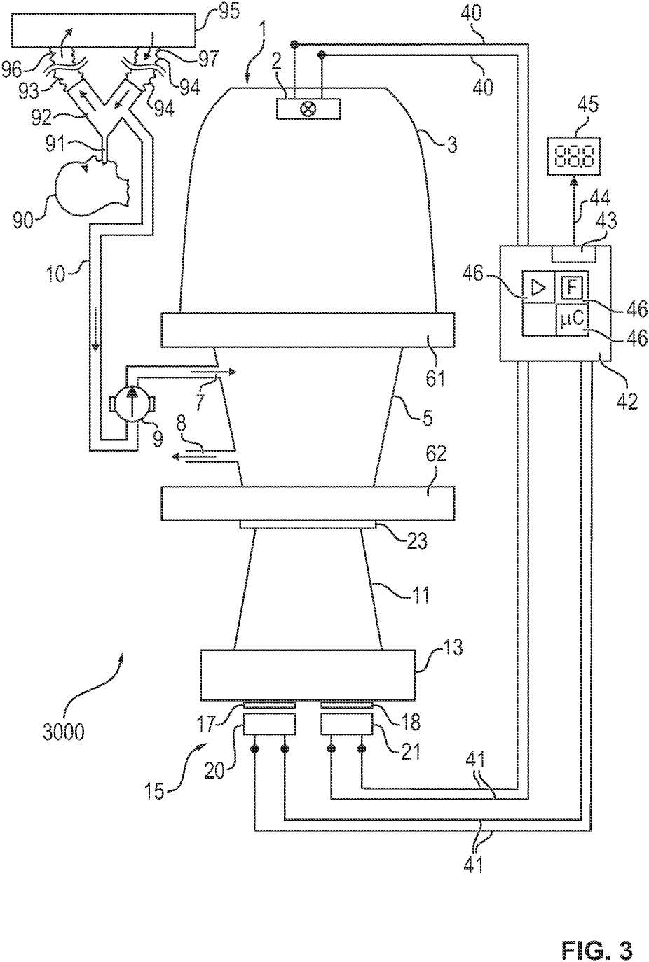

FIG. 3 shows a schematic representation of a gas measuring device 3000 according to a third embodiment. Therein, the same elements are designated with the same reference numerals as in FIGS. 1 and 2.

The third embodiment 3000 shown in FIG. 3 differs from the first embodiment shown in FIG. 1 in that an inlet silicon window 23 is disposed between the exit cell window 62 and the light guide element 11. The inlet silicon window 23 replaces a portion of the length 111, LA of the light guide element 11. In the present embodiment example, the inlet silicon window 23 has a thickness of 0.5 mm and thus replaces about 1.7 mm of the length 111, LA of the light guide element 11. In all other respects, the dimensions of the third embodiment example shown in FIG. 3 correspond to those of the first embodiment example shown in FIG. 1 with reference to the dimension designations in FIG. 7. In alternative embodiment examples, the inlet silicon window 23 may have a different thickness.

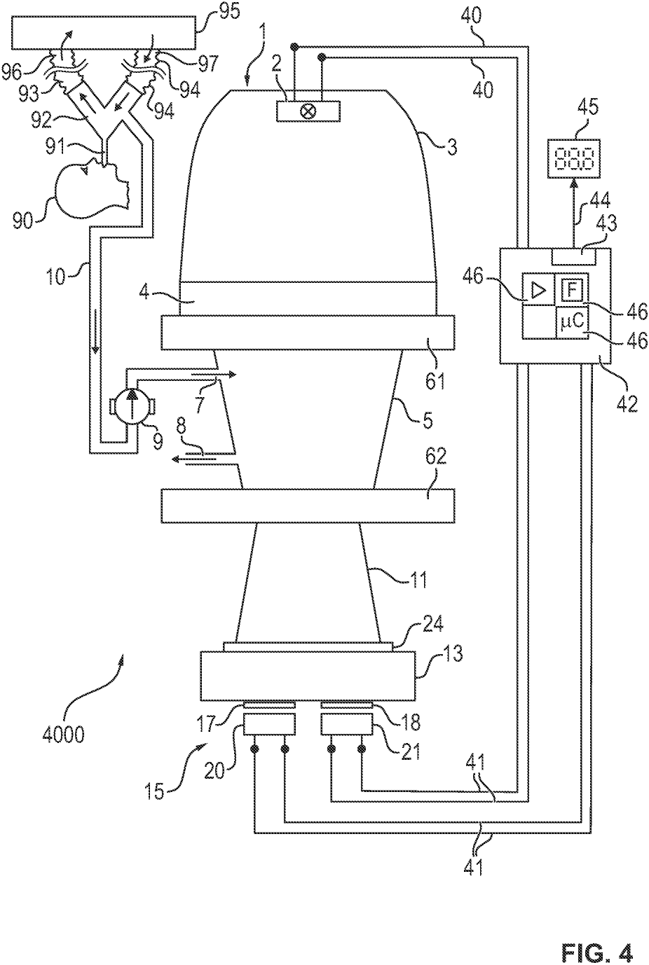

FIG. 4 shows a schematic representation of a gas measuring device 4000 according to a fourth embodiment. Here, the same elements are designated with the same reference numerals as in FIGS. 1 to 3. The gas measuring device 4000 according to FIG. 4 differs from FIG. 1 by an outlet silicon window 24, which is arranged between the light guiding element 11 and the entrance window 13 of the detector arrangement 15. The outlet silicon window 24 replaces a portion of the length 111, LA of the light guide element 11. In the present embodiment example, the outlet silicon window 24 has a thickness of 0.5 mm and thus replaces about 1.7 mm of the length 111, LA of the light guide element 11. In all other respects, the dimensions of the fourth embodiment example shown in FIG. 4 correspond to those of the first embodiment example shown in FIG. 1 with reference to the dimension designations in FIG. 7. In alternative embodiment examples, the outlet silicon window 24 may have a different thickness.

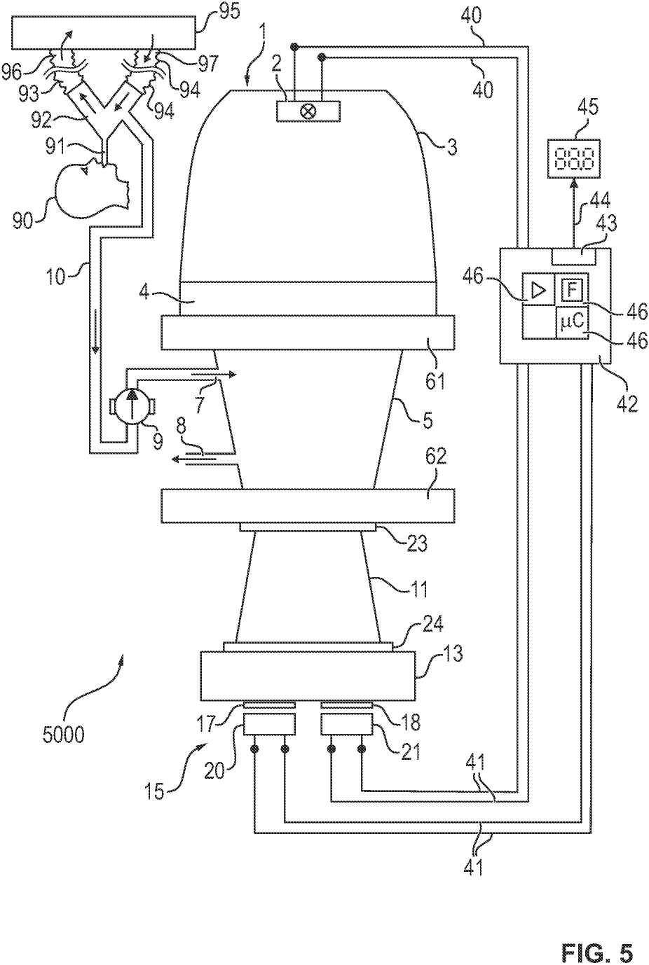

FIG. 5 shows a schematic representation of a gas measuring device 5000 according to a fifth embodiment. Therein, the same elements are designated with the same reference numerals as FIGS. 1 to 4. The gas measuring device 5000 according to FIG. 5 differs from FIG. 1 by the entry silicon window 23, which is arranged between the outlet cuvette window 62 and the light guiding element 11, and the outlet silicon window 24, which is arranged between the light guiding element 11 and the entry window 13 of the detector arrangement 15. The silicon windows 23, 24 replace a portion of the length 111, LA of the light guide element 11.

In this embodiment example, the cumulative thickness of the silicon windows 23, 24 is 1 mm, replacing about 3.4 mm of the length 111, LA of the light guide element 11. In all other respects, the dimensions of the fifth embodiment example according to FIG. 5 correspond to those of the first embodiment example according to FIG. 1 with reference to the dimension designations in FIG. 7. In alternative embodiment examples, the silicon windows 23, 24 may also have a different thickness.

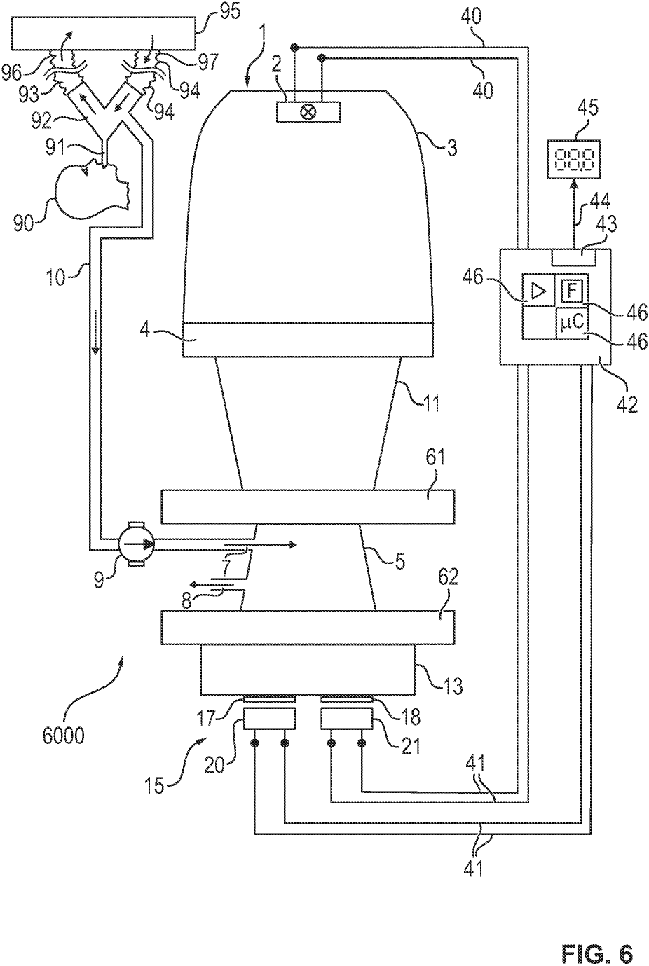

FIG. 6 shows, as a sixth embodiment 6000, a variant with radiation source 1, light guide element 11, sample gas cuvette 5, detector arrangement 15. The sixth embodiment 6000 according to FIG. 6 shows an arrangement of cuvette 5 and light guide element 11 in the beam path from radiation source 1 to detector arrangement 15 which is reversed with respect to FIGS. 1 to 5. The arrangement of light guide element 11 and sample gas cuvette 5 is configured in the reverse order to that in FIG. 1, following the light radiation and light propagation from radiation source 1 to detector arrangement 15. Identical elements of FIGS. 1 to 6 are designated with the same reference numerals as in FIGS. 1 to 5. The light radiation passes, starting from the radiation source 1, through the light guide element 11, the inlet cuvette window 61, the cuvette 5, the outlet cuvette window 62 and then reaches the detector arrangement 13.

The sixth embodiment 6000 according to FIG. 6 shows by way of example and based on the first embodiment 1000 according to FIG. 1 - but not restricted to FIG. 1, but rather also including embodiments according to FIGS. 2 to 5 in the sense of the invention — how a reversed sequence of light guide element 11 and sample gas cuvette 5 can be configured. The metrological functions and further aspects of the components shown in FIG. 6 result as described with respect to FIG. 1. In the embodiment example according to FIG. 6, the light radiation of the radiation source 1 passes successively through the exit window 4 of the radiation source 1, the light guide element 11, the inlet cuvette window 61, the sample gas cuvette 5, the outlet cuvette window 62, the entry window 13 of the detector arrangement 15, the bandpass filters 17, 18 and impinges on the detector elements 20, 21.

In alternative embodiments of the radiation source 1 with illuminant 2 and mirror arrangement 3 — as shown for example in FIG. 2 — the exit window 4 can be omitted.

In alternative embodiments of the detector arrangement 15, the entry window 13 may be omitted.

FIG. 7 shows a dimensioned schematic drawing of the configuration of the gas measuring devices according to FIGS. 1 to 6 with dimensions. The same elements of FIGS. 1 to 7 are designated with the same reference numbers as in FIGS. 1 to 6. Positions and dimensions of:
Length LC 31 of the mirror arrangement 3,
Outlet opening DC 35 of the mirror arrangement 3,
Length LB 51 of the sample gas cuvette 5,
Inlet opening DB1 52 of the sample gas cuvette 5,
Outlet opening DB2 53 of the sample gas cuvette 5,
Taper angle β 54 of the sample gas cuvette 5,
Length LA 111 of the light guide element 11,
Outlet opening DA1 112 of the light guide element 11,
Inlet opening DA2 113 of the light guide element 11,
Taper angle α 114 of the light guide element 11
shown.

The light guiding element 11 as well as the sample gas cuvette 5 are formed in a hollow truncated cone shape in FIGS. 1 to 7, other shapes formed as hollow bodies with round, elliptical, oval, triangular, square, polygonal or free-form shaped entry surfaces and exit surfaces of the light guiding element 11 or the sample gas cuvette with different sizes of entry surfaces and exit surfaces are included in the sense of the embodiments. For example, a truncated pyramid-shaped configuration may also be possible. FIGS. 1 to 7 show — also in the sense of simplified graphic representations —hollow frustoconical formations of the light guide element 11 as well as of the sample gas cuvette 5.

While specific embodiments of the invention have been shown and described in detail to illustrate the application of the principles of the invention, it will be understood that the invention may be embodied otherwise without departing from such principles.

List of reference Characters

| | |
|---|---|
| 1 | Radiation source with light guide |
| 2 | Illuminant, membrane spotlight, LED |
| 3 | Mirror arrangement |
| 4 | Emission window of the radiation source |
| 5 | Sample gas cuvette (hollow truncated cone) |
| 7 | Gas inlet |
| 8 | Gas outlet |
| 9 | Pump |
| 10 | Measuring gas line (sample line) |
| 11 | Light guide element (light guide, hollow truncated cone) |
| 12 | Aperture |
| 13 | Inlet window of the detector assembly |
| 15 | Detector arrangement |
| 17s | bandpass filter (measuring wavelength) |
| 18 | Second bandpass filter (reference wavelength) |
| 20 | Measurement detector |
| 21 | Reference detector |
| 23 | Entry silicon window |
| 24 | Outlet silicon window |
| 31 | Length LC of the mirror arrangement |
| 35 | Outlet opening DC of the mirror arrangement |
| 40 | Control lines, control signals |
| 41 | Measuring signal lines, measuring signals |
| 42 | Control unit |
| 43 | Interface |
| 44 | Output signal |
| 45 | Display unit |
| 46 | Components of signal processing |
| 51 | Length LB of the sample gas cuvette |
| 52 | Inlet opening DB1 of the sample gas cuvette |
| 53 | Outlet opening DB2 of the sample gas cuvette |
| 54 | Cone angle β of the sample gas cuvette |
| 61 | Entry cuvette window |
| 62 | Outlet cuvette window |
| 90 | Patient |
| 91 | Endotracheal tube |
| 92 | Connection element (Y-piece) |
| 93 | expiratory ventilation tube |
| 94 | inspiratory ventilation tube |
| 95 | Breathing system, ventilator |
| 96 | expiratory gas inlet |
| 97 | inspiratory gas outlet |
| 111 | Length LA of the light guide element |
| 112 | Outlet opening DA1 of the light guide element |
| 113 | Inlet opening DA2 of the light guide element |
| 114 | Cone angle α of the light guiding element |
| 1000 | Gas measuring device |
| 2000 | Gas measuring device |
| 3000 | Gas measuring device |
| 4000 | Gas measuring device |
| 5000 | Gas measuring device |
| 6000 | Gas measuring device |
| 7000 | Dimensional drawing for gas measuring devices 1000 - 6000 |

What is claimed is:

1. A gas measuring device for determining the concentration of a gas component in a breathing gas mixture of a living being, the gas measuring device comprising:
a radiation source adapted to emit light radiation in a wavelength range of 2.5 µm to 12.5 µm, the radiation source comprising a mirror arrangement and a light source;
a sample gas cuvette comprising a hollow body for receiving the respiratory gas mixture, the hollow body having a cross-section that varies over a length of the sample gas cuvette, the sample gas cuvette further comprising: an inlet cuvette window configured as an inlet of the light radiation into the sample gas cuvette; an outlet cuvette window configured as an outlet of the light radiation from the sample gas cuvette; a gas inlet configured to supply the breathing gas mixture into the sample gas cuvette; and a gas outlet configured to output the breathing gas mixture from the sample gas cuvette;
a light guide element comprising a hollow body, the light guide element having a cross-section that varies over a length of the light guide element;
a detector arrangement comprising at least two bandpass filter elements for filtering the light radiation and at least two detector elements for receiving the filtered light radiation; and
a control unit configured to detect signals from the at least two detector elements and to determine the concentration of the gas component in the breathing gas mixture from the signals from the at least two detector elements.

2. A gas measuring device according to claim 1, wherein the light guiding element or the sample gas cuvette or both the light guiding element and the sample gas cuvette are formed with reflective surfaces on an inside thereof.

3. A gas measuring device according to claim 1, wherein:
the sample gas cuvette extends so as to be tapered between the radiation source and the light guide element;
the light guide element extends so as to be expanded between the sample gas cuvette and the detector arrangement;
the light radiation from the radiation source passes through the inlet cuvette window into the sample gas cuvette, through the outlet cuvette window and through the light guide element to the detector arrangement.

4. A gas measuring device according to claim 1, wherein:
the light guide element extends so as to be tapered between the radiation source and the sample gas cuvette;
the sample gas cuvette extends so as to be expanded between the sample gas cuvette and the detector arrangement;
the light radiation from the radiation source passes through the light guide element through the inlet cuvette window into the sample gas cuvette, through the outlet cuvette window to the detector arrangement.

5. A gas measuring device according to claim 1, wherein a length of the light guiding element is a value in a range of 2.5 mm to 5.0 mm.

6. A gas measuring device according to claim 1, wherein the sample gas cuvette and the light guide element are formed as a truncated pyramid.

7. A gas measuring device according to claim 1, wherein the sample gas cuvette, or the light guiding element or both the sample gas cuvette, or the light guiding element are formed as a truncated pyramid or as a truncated cone.

8. A gas measuring device according to claim 1, wherein the sample gas cuvette and the light guiding element are formed as a truncated cone.

9. A gas measuring device according to claim 7, wherein a pyramid angle or a cone angle of the light guiding element is a value in a range 7.5° to 20.0°.

10. A gas measuring device according to claim 7, wherein an inlet opening of the light guiding element is a value in a range 2.0 mm to 4.0 mm.

11. A gas sensing device according to claim 7, wherein one or more of:
a pyramid angle or a cone angle of the light guiding element is formed smaller than or equal to a pyramid angle or cone angle of the sample gas cuvette, and./or is defined by a ratio of the pyramid angle or the cone angle of the light guiding element to the pyramid angle or the cone angle of the sample gas cuvette having a value in a range of 0.75 to 1.0;
the length of the light guide element is made shorter than or equal to the length of the sample gas cuvette and/or is defined by a ratio of the length of the light guide element to the length of the sample gas cuvette having a value in a range of 0.3 to 1.0;
an outlet opening of the sample gas cuvette is larger than or equal to an inlet opening of the light guide element and/or is defined by a ratio of the outlet opening of the sample gas cuvette to the inlet opening of the light guide element having a value in a range from 1.0 to 2.0;
an inlet opening of the light guide element is formed smaller than the outlet opening of the light guide element and/or is defined by a ratio of the inlet opening of the light guide element to the outlet opening of the light guide element having a value in a range of 0.3 to 0.9;
an inlet opening of the sample gas cuvette is made larger than an outlet opening of the sample gas cuvette and/or is defined by a ratio of the inlet opening of the sample gas cuvette to the outlet opening of the sample gas cuvette having a value in a range from 1.2 to 1.8;
an outlet opening of the mirror arrangement is made larger than or equal to an inlet opening of the sample gas cuvette and/or is defined by a ratio of the outlet opening of the mirror arrangement to the inlet opening of the sample gas cuvette having a value in a range from 1.0 to 2.0; and
a ratio of the outlet opening of the mirror assembly to a length of the mirror assembly is a value in a range from 0.53 to 1.88.

12. A gas measuring device according to claim 1, wherein an inlet silicon window is arranged on a light inlet side of the light guiding element.

13. A gas measuring device according to claim 1, wherein an outlet silicon window is arranged on the light guide element on a light exit side.

14. A gas measuring device according to claim 1, wherein:
an inlet silicon window is arranged on a light inlet side of the light guiding element;
an outlet silicon window is arranged on the light guide element on a light exit side; and
an optical path outside the sample gas cuvette and/or a length of the light guiding element is reduced by a value in a range of 2.0 to 4.0 times a cumulative thicknesses of the silicon windows.

15. A gas measuring device according to claim 1, wherein:
an inlet silicon window is arranged on a light inlet side of the light guiding element;
an outlet silicon window is arranged on the light guide element on a light exit side; and
a ratio of the length of the light guiding element to the length of the sample gas cuvette is reduced according to the cumulative thicknesses of the silicon windows.

16. A gas measuring device according to claim 1, wherein:
an inlet silicon window is arranged on the light guiding element on a light inlet side; and
the inlet silicon window replaces a portion of the light guiding element and a thickness of the inlet silicon window is less than the replaced portion of the light guiding element.

17. A gas measuring device according to claim 1, wherein:
an outlet silicon window is arranged on the light guide element on a light exit side; and
the outlet silicon window replaces a portion of the light guiding element and a thickness of the outlet silicon window is less than the replaced portion of the light guiding element.
